(19) United States
(12) Patent Application Publication
(10) Pub. No.: US 2009/0054250 A1
(43) Pub. Date: Feb. 26, 2009

(54) Methods to create fluorescent biosensors using aptamers with fluorescent base analogs

(76) Inventors: Evaldas Katilius, Phoenix, AZ (US); Zivile Katiliene, Phoenix, AZ (US); Neal W. Woodbury, Tempe, AZ (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/664,914

(22) PCT Filed: Nov. 2, 2005
(86) PCT No.: PCT/US05/39852

§ 371 Date: Nov. 6, 2008

(51) Int. Cl. C40B 30/04; C07H 1/00; C07H 21/00; C40B 40/08

(52) U.S. Cl. 506/9; 536/25.3; 536/24.3; 506/17

(57) ABSTRACT

The present invention provides improved methods for generating fluorescent aptamer polynucleotides, novel polynucleotides, and methods for use thereof.

### CROSS REFERENCE

This application claims priority to U.S. Provisional Patent Application Ser. No. 60/625,247 filed Nov. 4, 2004, which is incorporated by reference herein in its entirety.

### STATEMENT OF GOVERNMENT SUPPORT

Financial assistance for this project was provided by the U.S. Government, National Science Foundation #MPS-0239986; thus, the United States Government has certain rights to this invention.

### FIELD OF THE INVENTION

The invention relates to the fields of nucleic acids, proteins, binding interactions between nucleic acids and proteins, and bioassays.

### BACKGROUND

An aptamer is a synthetic oligonucleotide designed to bind to a ligand of interest, often a protein. Various selection techniques, such as SELEX (systematic evolution of ligands through exponential enrichment), have been designed to identify aptamers with optimal binding characteristics for a ligand of interest. Such aptamers have varied potential uses, including as therapeutics and for detecting the ligand of interest. Where detection of the ligand is desired, the aptamer must not only bind to the ligand, but the interaction must be detectable.

Fluorescent DNA base analogs have been used in studies of DNA base dynamics. The direct application of these molecules for detecting DNA oligonucleotide hybridization is described, for example, in U.S. Pat. No. 6,451,530. However, the use of fluorescent base analogs for hybridization applications is based on DNA base pairing and creating a mismatch leading to a base bulge. In contrast, the use of fluorescent base analogs for detection of aptamer binding to its ligand is based on the tertiary structures of aptamers and conformational changes in the aptamer due to ligand binding.

### SUMMARY OF THE INVENTION

In one aspect, the present invention provides improved method for generating fluorescent aptamer polynucleotides, wherein the improvement comprises synthesizing the aptamer sequence with at least one aptamer nucleotide replaced by a fluorescent base analog, wherein the fluorescence intensity of the modified aptamer is detectably altered upon binding to its ligand.

In another aspect, a polynucleotide, comprising a nucleic acid sequence selected from the group consisting of

| | | |
|---|---|---|
| (a) | 5′-GGTTGGXGTGGTTGG-3′; | (SEQ ID NO: 1) |
| (b) | 5′ GGG GCA CGT TTA TCC GTX CCT CCT AGT GGC GTG CCC | (SEQ ID NO: 2) |
| | C 3′; | |
| and | | |
| (c) | 5′ CAC AGG CTA CGG CAC GTA GAG XAT CAC CAT GAT CCT | (SEQ ID NO: 3) |
| | GTG 3′, | |

wherein X is a fluorescent base analog.

In a further aspect, a method to detect a ligand of interest, comprising

(a) contacting a sample to be tested with one or more polynucleotides of the invention under conditions to promote binding of the one or more polynucleotides to their relevant ligand;

(b) detecting fluorescence from the one or more polynucleotides; and

(c) correlating an altered fluorescence with the presence of the ligand in the test sample.

### DESCRIPTION OF THE FIGURES

FIG. 1. Fluorescence spectra of thrombin aptamer labeled with 4-amino-6-methyl-isoxanthopterin (6MAP) at the position 7 before (dashed line) and after addition of human α-thrombin protein (solid line). The relative increase in fluorescence upon protein binding is about 30-fold.

FIG. 2. a) Secondary structure of the IgE aptamer calculated using the DNA mfold program [3]. b) Twelve substitutions of bases in the loop region with 2-aminopurine were screened for fluorescence increase upon protein addition and a positive response was obtained for position #18. c) PAGE purified aptamer with 2-aminopurine at the position 18 shows 5.6-fold fluorescence increase upon binding saturation.

FIG. 3. a) Two secondary structure conformations of the PDGF-B aptamer calculated using mfold. b) Five aptamers modified with 2AP were screened for signaling effect, position #22 has shown significant conformation change.

### DETAILED DESCRIPTION OF THE INVENTION

All publications, patents and patent applications cited herein are hereby expressly incorporated by reference for all purposes.

Within this application, unless otherwise stated, the techniques utilized may be found in any of several well-known references such as: Molecular Cloning: A Laboratory Manual (Sambrook, et al., 1989, Cold Spring Harbor Laboratory Press) and PCR Protocols: A Guide to Methods and Applications (Innis, et al. 1990. Academic Press, San Diego, Calif.).

In one aspect, the present invention provides improved methods for generating fluorescent aptamer polynucleotides, wherein the improvement comprises synthesizing the aptamer sequence with at least one nucleotide replaced by a fluorescent base analog, wherein the fluorescence intensity of the modified aptamer is detectably increased or decreased upon aptamer binding to ligand molecule. As used herein, the term “ligand” includes proteins, lipids, carbohydrates, nucleic acids, or other molecules, but specifically excludes any nucleic acid ligand that is bound via complementary base-pairing.

As used herein, the term “detectably increased or decreased” means any difference between the unbound modified aptamer and the modified aptamer when bound to its ligand that can be detected using standard detection techniques. In various preferred embodiments, the increase or decrease in fluorescence intensity upon binding of the modified aptamer to its ligand is at least 5%, 10%, 20%, 25%, 50%, 75%, 100%, 200%, 300%, 400%, 500%, or 1000% greater or less than the corresponding fluorescence intensity of the unbound modified aptamer.

The fluorescent aptamer polynucleotides can be RNA or DNA, and can be single or double stranded. In a preferred embodiment of the methods of the invention, the aptamers are 10-80 nucleotides in length. Methods for identifying aptamer sequences to bind to a ligand of interest, such as SELEX, are known in the art. (See, for example, U.S. Pat. Nos. 5,475,096 and 5,270,163, one of refs.: Gold, L., et al., Diversity of oligonucleotide functions. Annu Rev Biochem, 1995. 64: p. 763-97.)

Selection of appropriate fluorescent aptamer polynucleotides according to the methods of the invention can be performed after selection of aptamers for a given ligand using SELEX or any other selection technique. In a preferred embodiment of the methods of the invention, the selection of appropriate fluorescent aptamer polynucleotides is performed after synthesis of a pool of the aptamers with fluorescent nucleotide analogs. Placement of fluorescent base analogs in the DNA or RNA sequence can be random, thus testing all possible positions of the sequence for the best response to the ligand binding. Another approach for the selection is to limit the number of possible replacements after secondary (or even more preferably, tertiary) structure inspection. Software packages for secondary structure predictions for DNA or RNA sequences are readily available and well known to those skilled in art. Some examples include DNA mfold (http://www.bioinfo.rpi.edu/applications/mfold/old/dna/), RNAfold (http://ma.tbi.univie.ac.at/cgi-bin/RNAfold.cgi), GeneBee (http://www.genebee.msu.su/services/rna2_full.html), RNAsoft (www.rnasoft.ca) and others. Secondary structure predictions usually effectively predict the base paired regions of DNA or RNA oligonucleotides (aptamers), while the binding site for the ligand is usually located in the unstructured region of oligonucleotide. Thus, this reduces the number of possible base analog replacements and amounts of samples to be tested. In an even more preferred approach, the particular positions for fluorescent nucleotide incorporation can be determined from the tertiary (crystal or NMR) structure of aptamer and aptamer bound to the ligand. This approach would directly reveal the positions where DNA or RNA bases undergo significant conformation rearrangement upon ligand binding, which then can be tested by replacing the bases with fluorescent nucleotides and inspecting the fluorescence changes upon ligand binding to the aptamer. However, this approach is limited by the difficulties in obtaining structures with atomic resolution.

“Fluorescent nucleotide” or “fluorescent base analog” is a nucleotide or nucleotide analogue that is capable of producing fluorescence when excited with light of an appropriate wavelength. The fluorescence signal is greatly reduced or eliminated when the nucleotide is incorporated into an oligonucleotide and undergoes base stacking with neighboring bases. However, as long as the nucleotide analog fluoresces with a quantum yield above 0.04, more preferably above 0.1 and most preferably above 0.15 when it exists as a monomer in an aqueous solution it is regarded as a fluorescent nucleotide. Fluorescent nucleotides include, but not limited to, 2-amino purine (2AP), 3-methyl-isoxanthopterin (3MI), 6-methylisoxanthopterin (6MI), 4-amino-6-methyl-pteridone (6MAP), 4-amino-2,6-dimethyl-pteridone (DMAP), pyrrolo-dC, 5-methyl-2-pyrimidone.

Fluorescent aptamer polynucleotides generated according to the above methods can be contacted with a test sample thought to contain the ligand of interest under any type of conditions suitable for the desired binding event. Examples of test samples include, but are not limited to, purified ligand, ligand mixtures, cell lysates, cell culture medium, protein extracts, tissue samples, pathology samples, bodily fluid samples including but not limited to blood, urine, semen, saliva, vaginal secretions, and sweat. Appropriate conditions for promoting binding of the fluorescent aptamer polynucleotide and the ligand of interest within the test sample can be determined using routine methods by those of skill in the art.

Any means in the art for detecting fluorescence from the fluorescent aptamer polynucleotides upon binding to the ligand of interest can be used, including but not limited to fluorescence spectrometers, fluorescence microscopes, fluorescent plate readers, fluorescence (DNA chip) scanners or imagers, and others.

Existing technologies allow the generation of specific aptamers for nearly any ligand of interest. The methods disclosed herein provide the ability to generate a fluorescent biosensor to a ligand of interest. When combined with other existing technologies to make DNA microarrays, the methods can be used to generate fluorescent aptamer polynucleotides for the manufacture of high-throughput (HT) detection systems, or to generate novel imaging agents for in vivo biological imaging. The main advantage of the methods disclosed herein over previously presented approaches is that the fluorescent signals generated from the fluorescent aptamer polynucleotides made according to the methods of the invention are binding specific, and generate signals with much better signal to noise ratios comparing to previously described methods, as in S. Jhaveri, M. Rajendran, A. D. Ellington, Nature Biotechnology 18, 1293-1297 (December, 2000); S. D. Jhaveri et al., Journal of the American Chemical Society 122, 2469-2473 (Mar. 22, 2000). Another advantage of generating signal using incorporated fluorescent nucleotides is that samples to be tested do not need to be labeled, which simplifies testing protocols.

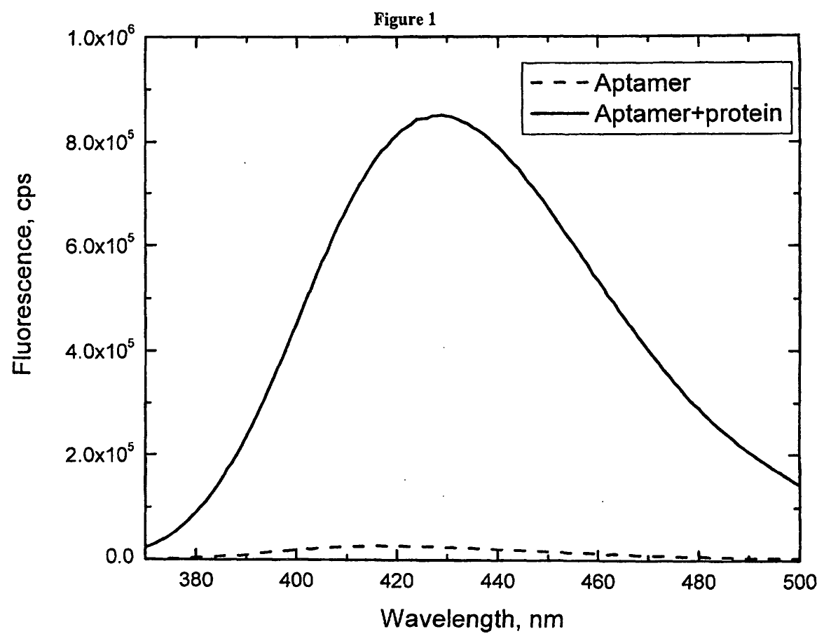

In a second aspect, the present invention provides novel polynucleotides, comprising or consisting of the sequence of SEQ ID NO: 1, 5′-GGTTGGXGTGGTTGG-3′, wherein “X” is defined as a fluorescent base analog. This novel polynucleotide is shown herein to serve as a fluorescent biosensor for human α-thrombin protein, wherein the biosensor fluorescence is greatly increased upon binding to the human α-thrombin protein, and wherein its fluorescence is significantly quenched in the absence of binding to the human α-thrombin protein (see, for example, FIG. 1). Such an α-thrombin biosensor can be used for any method wherein detection of α-thrombin is desired, including but not limited to those described below.

In a third aspect, the present invention provides novel polynucleotides, comprising or consisting of the sequence of SEQ ID NO:2, 5′ GGG GCA CGT TTA TCC GTX CCT CCT AGT GGC GTG CCC C 3′, wherein “X” is defined as a fluorescent base analog. This novel polynucleotide is shown herein to serve as a fluorescent “biosensor” for human IgE immunoglobulin (“IgE”), wherein the biosensor fluorescence is greatly increased upon binding to IgE, and wherein its fluorescence is significantly quenched in the absence of binding to IgE. Such an IgE biosensor can be used for any method wherein detection of IgE is desired, including but not limited to those described below.

In a fourth aspect, the present invention provides novel polynucleotides, comprising or consisting of the sequence of SEQ ID NO:3, 5′CAC AGG CTA CGG CAC GTA GAG XAT CAC CAT GAT CCT GTG 3′, wherein “X” is defined as a fluorescent base analog. This novel polynucleotide is shown herein to serve as a fluorescent “biosensor” for human platelet-derived growth factor B (“PDGF-B”), wherein the biosensor fluorescence is greatly increased upon binding to PDGF-B, and wherein its fluorescence is significantly quenched in the absence of binding to PDGF-B. Such a PDGF-B biosensor can be used for any method wherein detection of PDGF-B is desired, including but not limited to those described below.

The polynucleotides of the second, third, and fourth aspects of the invention can be single or double stranded. In a preferred embodiment, the polynucleotide is between 15 and 80, 15-70, 15-60, 15-50, 15-40, 15-30, or 15-25 nucleotides in length.

Fluorescent base analogs that can be used in the polynucleotide of the invention include, but are not limited to 2-amino purine (2AP), 3-methylisoxanthopterin (3MI), 6-methylisoxanthopterin (6MI), 4-amino-6-methyl-pteridone (6MAP), 4-amino-2,6-dimethyl-pteridone (DMAP), pyrrolo-dC, 5-methyl-2-pyrimidone.

The polynucleotides of the invention can comprise one or more other chemical groups to provide desired properties, including but not limited to other fluorescent molecules, affinity tags (including but not limited to biotin, digoxygenin, and fucose); fluorescent beads or fluorescent quantum dots, and reactive groups/linkers (including but not limited to amino, carboxy or thiol reactive group (either 5′, 3′ or on the internal base) to provide for further binding and/or signaling functionality on the polynucleotide.

Fluorescent nucleotide analogs used for polynucleotide modification (as in the polynucleotides of the present invention) fluoresce in the spectral range from 350 to 500 nm. This limits the ability to create multiplex assays using multiple solution suspended polynucleotides. Thus, in another embodiment, the polynucleotides of the invention further comprise one or more reference fluorophores. For example, one can use Forster resonance energy transfer (also called Fluorescence Resonance Energy Transfer, or FRET) to shift the fluorescence response toward the longer wavelengths. FRET is a physical phenomenon, when two fluorescent molecules, usually called donor and acceptor, interact when sufficiently close to each other (usually less that 10 nm apart). This interaction causes the energy from the excited donor molecule to be transferred to acceptor molecule and as a result, the acceptor molecule becomes excited and fluoresces, while fluorescence of the donor molecule is quenched. FRET requires that the fluorescence spectrum of the donor molecule and absorbance spectrum of the acceptor molecule overlap. The theory and practical implementations of FRET are well known to those skilled in art.

FRET based fluorescence signal transduction mechanism can be extended further, as the fluorescent molecule, which accepts the energy from the fluorescent nucleotide analog, can act as donor and transfer the energy to another fluorescent molecule which has an appropriate spectral properties to act as an acceptor molecule, and so on. The implementation of FRET based fluorescence signal transfer cascade is understood by those skilled in art.

Preferred fluorescence energy acceptor molecules (ie: one example of reference fluorophores) for use with the polynucleotides of the invention depend on the particular fluorescent nucleotide used as energy donor molecule. In the case of 3MI, 6MI, 6MAP or DMAP, preferred fluorescence energy acceptor molecules include, but are not limited to, AlexaFluor™ 430, Lucifer Yellow™ CF, Acridine yellow, pyMPO (1-(3-carboxybenzyl)-4-(5-(4-methoxyphenyl)oxazol-2-yl) pyridinium bromide)), NBD-X. In case of 2AP, preferred acceptor molecules include, but are not limited to, Cascade Blue™, 7-methoxycoumarin, Prodan, stilbene, Marina Blue™, dimethylaminocoumarin. These fluorescent molecules can be obtained from various commercial sources including, but not limited to, Invitrogen Corporation, GE Healthcare, Sigma Aldrich.

Thus, when one or more reference fluorophores are included in the polynucleotides of the invention to generate FRET, the reference fluorophores are chosen to have an absorbance spectrum overlapping with the fluorescence spectrum of the fluorescent nucleotide incorporated into the polynucleotide. The reference fluorophores, acting as acceptors, are chosen so that the distance between fluorescent nucleotide (donor) and fluorescent molecule (acceptor) is within the distance required to observe FRET between the two molecules, as is well known to those of skill in the art. As a result, FRET between the fluorescent nucleotide and the reference fluorophores results in the fluorescence of the acceptor molecule appearing or increasing when fluorescent nucleotide is excited. As the intensity of the nucleotide analog fluorescence increases to report the binding of aptamer to its ligand, fluorescence of the acceptor molecule also increases as a result of FRET.

When the polynucleotides of the invention further comprise a reference fluorophores that comprises an affinity tag, such as a biotin tag, the polynucleotides can be allowed to interact with streptavidin, avidin or neutravidin (or any other biotin binding protein) which are fluorescently labeled with the appropriate acceptor molecule. Then, the interaction between the fluorescent nucleotide present in the polynucleotides of the invention and the acceptor molecule present on the surface of streptavidin (or other protein) can lead to FRET, and as a result fluorescence of the acceptor molecule will report binding of the polynucleotides of the invention to their ligand through increase in intensity.

The polynucleotides of the invention can be provided in lyophilized form, in solution, or bound to a solid support. Such solid supports are those that permit conformational changes to the polynucleotide upon binding to its ligand. Examples of such solid surface materials include, but are not limited to, microarrays, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose, polyacrylamides, methylmethracrylate polymers; sol gels; porous polymer hydrogels; nanostructured surfaces; nanotubes (such as carbon nanotubes), and nanoparticles (such as gold nanoparticles or quantum dots). The solid surfaces can comprise one or a plurality of immobilized polynucleotides of the invention. When bound to a solid support, the polynucleotides can be directly linked to the support, or attached to the surface via a linker. Thus, the solid support surface and/or the polynucleotide can be derivatized using methods known in the art to facilitate binding of the polynucleotide to the solid support, so long as the derivitization does not eliminate detection of binding between the polynucleotide and its relevant ligand. Other nucleic acids, such as reference or control nucleic acids, can be optionally immobilized on the solid surface as well. Methods for immobilizing nucleic acids on a variety of solid surfaces are well known to those of skill in the art. A wide variety of materials can be used for the solid surface.

A variety of different materials may be used to prepare the solid surface to obtain various properties. For example, proteins (e.g., bovine serum albumin) or mixtures of macromolecules (e.g., Denhardt's solution) can be used to minimize non-specific binding, simplify covalent conjugation, and/or enhance signal detection. If covalent bonding between a compound and the surface is desired, the surface will usually be functionalized or capable of being functionalized. Functional groups which may be present on the surface and used for linking include, but are not limited to, carboxylic acids, aldehydes, amino groups, cyano groups, ethylenic groups, hydroxyl groups, and mercapto groups. Methods for linking a wide variety of compounds to various solid surfaces are well known to those of skill in the art.

In a fifth aspect, the present invention provides methods to detect a ligand of interest, comprising contacting a sample to be tested with one or more polynucleotides of the invention under conditions to promote binding of the one or more polynucleotides with their relevant ligand, detecting fluorescence from the one or more polynucleotides, and correlating an altered fluorescence with the presence of the ligand in the test sample.

In various preferred embodiments, the method can further comprise removing unbound test sample and/or polynucleotide by, for example, adding a wash step (preferable when the one or more polynucleotides are bound to a solid support); multiple rounds of contacting and washing steps; and comparing the fluorescence emitted from the one or more polynucleotides in comparison to control, such as unbound polynucleotide or polynucleotide contacted with a test sample known to not contain the ligand of interest, for example.

The one or more polynucleotides can be contacted with a test sample thought to contain the ligand of interest under any type of conditions suitable for the desired binding event. Examples of test samples include, but are not limited to, purified ligand or ligand mixtures, cell lysates, cell culture medium, environmental samples (for example, water for human or agricultural consumption, water in lakes, streams, etc, effluent from fabrication facilities), protein extracts, tissue samples, pathology samples, bodily fluid samples including but not limited to blood, urine, semen, saliva, vaginal secretions, and sweat, samples collected from surfaces or air and suspended in water (applications such as biothreat detection, or other contamination issues), and animal samples, such as samples collected from agricultural livestock or from agricultural products (chicken carcass wash water, brain, blood, meat, milk or egg testing, for example).

Appropriate conditions for promoting binding of the fluorescent aptamer polynucleotide and the ligand of interest within the test sample can be determined using routine methods by those of skill in the art.

The methods can be conducted in solution or on a solid support, as disclosed above. For example, the one or more polynucleotides can be bound to a column or “dipstick” apparatus and a liquid test sample passed over the solid support. Alternatively, one or more polynucleotides according to the invention can be immobilized on the end of an optical fiber(s) to provide an integrated system for ligand detection in situ. In a further embodiment, polynucleotide(s of the invention) can be embedded in a wipe (preferably a moist wipe) for wiping on a surface, and then fluorescence signal (and ligand) can be detected from the wipe. In a further embodiment arrays of polynucleotides on solid supports are provided, as discussed above, to permit multiplex testing. Yet another embodiment comprises immobilization of polynucleotides on the surfaces of beads and using these beads to interact with a test sample, and then flow through the beads through a capillary electrophoresis or microfluidic device (‘Lab-on-a-chip’ device) to detect the presence of the target molecules in the test samples.

Any means in the art for detecting fluorescence from the one or more polynucleotides upon binding to the ligand of interest can be used, including but not limited to fluorescence spectrometers, fluorescence microscopes, fluorescence multi-channel plate readers, fluorescence (DNA chip) scanners or imagers, fluorescence activated cell sorters, capillary electrophoresis/laser-induced fluorescence devices, epifluorescent systems including those employing optical fibers and others.

It is often the case that one wants a quantitative estimate of the number of aptamers that have a ligand bound. Such information provides direct measurements of ligand concentration rather than just presence or absence of ligand. Quantitating levels of the ligand can be accomplished using fluorescence analysis. For example, fluorescence signals from polynucleotides can be calibrated using known concentrations of ligand, and this calibration curve can be used for quantification of ligand concentration. However, it is sometimes impractical to control exactly how much aptamer is present in the sample with high accuracy, making such quantitative ligand binding measurements difficult or inaccurate. This problem can be overcome by the attachment of a reference fluorophore to the polynucleotides that does not interact (for example via FRET as discussed herein) with the fluorescent base analog, but serves as a reference fluorescent signal at a different wavelength of emission than the fluorescent base analog. Comparison of the reference signal with the signal from the fluorescent base analog makes it possible to determine directly the fraction of the aptamer molecules with ligand bound without having had to externally determine the total number of aptamer molecules present. Preferred reference fluorescent molecules include Rhodamine dyes (e.g., R-560, R-575 R-590, R-610, sulphorhodamine, Kiton Red, etc), DCM, LDS dyes, Oxazine dyes, etc. emitting well to the red of the fluorescent base analogs currently available (these dyes are described and sold by, for example, Exciton Inc. or Invitrogen Corporation). Another preferred fluorescent addition would be a quantum dot, as this could be chosen to fluorescence well to the red of the fluorescent base analogs but be excited by the same laser wavelength, simplifying the experimental setup required for detecting fluorescence from both fluorophores

In one embodiment of this fifth aspect, the one or more polynucleotides comprise or consist of the α-thrombin fluorescent aptamer (SEQ ID NO:1), and the ligand is human α-thrombin. In this embodiment, the methods can be used in any assay where detection of α-thrombin is desirable, for example, assessing appropriate levels of hemostasis in a subject (ie: assessing thrombin formation capacity of plasma samples), which reflects the ability of the subject's hemostatis system to control bleeding (US 20050221414). Such methods can be used, for example, to assess a subject's ability to control bleeding in response to certain therapeutic treatments, and thus are useful to monitor efficacy and safety of the treatment.

In another embodiment of this fifth aspect, the one or more polynucleotides comprise or consist of the IgE fluorescent aptamer (SEQ ID NO:2), and the ligand is IgE. In this embodiment, the methods can be used in any assay where detection of IgE is desirable, for example assessing immunological dysfunction in a subject. For example, IgE blood levels are increased in subjects that have come in contact with an allergen compared to subjects not allergic to the allergen (US 2005176067). Thus, the methods of this embodiment can be used, for example, to assess whether a subject has an allergy and can be used to define the allergen. In one embodiment, a specific allergen can be bound to a column and a serum sample from the subject run over the column to provide for IgE binding to the allergen, followed by contacting with the IgE biosensor of SEQ ID NO:2; binding of the biosensor to the IgE can then be detected as discussed above.

In a further embodiment of this fifth aspect, the one or more polynucleotides comprise or consist of the PDGF-B fluorescent aptamer (SEQ ID NO:3), and the ligand is PDGF-B. In this embodiment, the methods can be used in any assay where detection of PDGF-B is desirable, for example assessing atherosclerosis and tumor risk in a subject. For example, PDGF B expression has been reported in vascular tissues involved in atherosclerosis, as well as in mesenchymal-appearing intimal cells and endothelial cells, respectively, of atherosclerotic plaques (US 20020094546). PDGF B has also been reported to be mitogen for cells of mesenchymal origin, and has been implicated in autocrine growth stimulation in the pathologic proliferation of endothelial cells characteristically found in glioblastomas.

The methods of the fifth aspect of the invention can also be used to identify compounds that bind to the ligand of interest, by, for example:

(a) contacting a polynucleotide according to claim 5 with its ligand under conditions to promote binding of the polynucleotide to the ligand to form a polynucleotide-ligand complex;

(b) detecting fluorescence from the polynucleotide-ligand complex;

(c) contacting the polynucleotide-ligand complex with candidate ligand binding compounds; and

(d) detecting fluorescence of the polynucleotide, wherein a change in fluorescence of the polynucleotide following step (c) relative to fluorescence detected from the polynucleotide-ligand complex correlates with the presence of a ligand binding compounds among the candidate ligand binding compounds.

Preferably, the change in fluorescence is a decrease in fluorescence. This is particularly useful for designing high-throughput assays, such as competition assays, to screen for inhibitors or possible drugs which would bind to the ligand of interest with higher affinity than the polynucleotide aptamers of the invention.

### EXAMPLE 1

### Fluorescent Human α-Thrombin Aptamer

A fluorescent aptamer for the human α-thrombin protein was generated, with the sequence 5′-GGTTGGTGTGGTTGG-3′ (SEQ ID NO:4). (L. C. Bock, L. C. Griffin, J. A. Latham, E. H. Vermaas, J. J. Toole, Nature 355, 564-566 (Feb. 6, 1992)) This aptamer was modified by replacing the thymine (“T”) at position 7 with 6MAP using standard synthetic solid-state DNA synthesis techniques. Fluorescence of 6MAP is strongly dependent on the media surrounding this molecule. Its fluorescence is strongly quenched when 6MAP is base stacked within the single or double stranded DNA aptamer. Unstacking of 6MAP within the aptamer upon binding to α-thrombin leads to nearly 33-fold increase in its fluorescence (see FIG. 1 and table 1). Thus, incorporation of 6MAP at this location leads to a large increase in fluorescence signal of polynucleotide when it binds to its ligand.

The example was reproduced using another fluorescent base analog, 2-aminopurine. Incorporation of 2-aminopurine into the aptamer of SEQ ID NO:2 at position 7 (replacing the “T” residue) as described above, leads to 10-fold increase in 2-aminopurine fluorescence upon aptamer binding to human α-thrombin. The same relative signal increase (nearly 10-fold) is also detected when aptamer is modified with 3-methylisoxanthopterin (see table 1).

TABLE 1

| | Fluorescence signal increase when aptamer is contacted | | |
|---|---|---|---|
| | with binding saturating concentration of target protein | | |
| Modified | | | |
| fluorescent | | 3- | 4-amino-6- |
| aptamer | 2-aminopurine | methylisoxanthopterin | methyl-pteridone |
| Thrombin | 10 | 9.7 | 32.9 |
| IgE | 5.6 | 1.5 | 1.9 |
| PDGF | 5.8 | 2.5 | 6.7 |

### EXAMPLE 2

### Fluorescent IgE Immunoglobulin Aptamer

In this example, a signaling aptamer of IgE immunoglobulin was made using secondary DNA structure calculation algorithms to minimize the set of possible modifications for screening.

The consensus sequence of IgE aptamer published in [1] is:

(SEQ ID NO: 5)

5′ GGG GCA CGT TTA TCC GTC CCT CCT AGT GGC GTG CCC
C 3′.

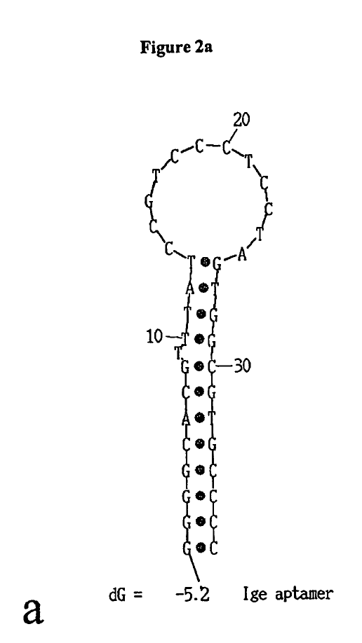

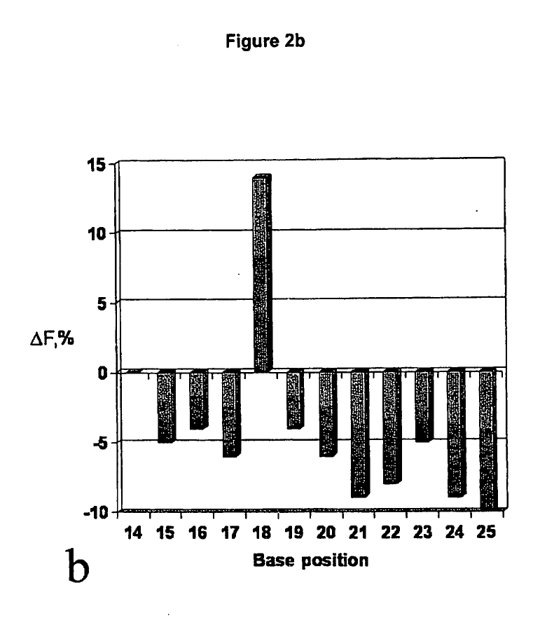

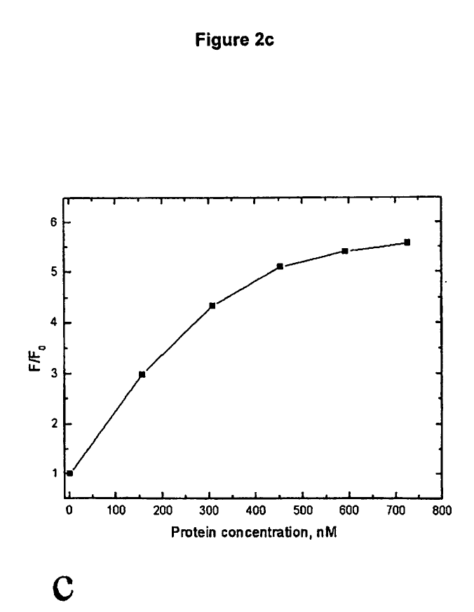

The secondary structure calculation for this aptamer is presented in FIG. 2a, which suggests that the aptamer folds into a hairpin conformation with a 14 base pair stem and 12 bases loop. Based on the calculated aptamer secondary structure, it can be hypothesized that the IgE immunoglobulin binding site consists mostly of loop nucleotides. Therefore, screening of the loop bases for signaling effect was performed using 2-aminopurine as a signaling molecule. Twelve different aptamers were synthesized, in which each of the bases in the loop region were replaced with 2-aminopurine. Screening was performed on unpurified aptamers (DNA oligonucleotides were desalted after synthesis) in physiological buffer (20 mM Tris-HCl pH 7.1, 140 mM NaCl, 5 mM KCl, 10 mM CaCl₂, 10 mM MgCl₂) at room temperature (23° C.). Fluorescence of aptamer solutions at 1 μM was measured before and after addition of IgE protein at 160 nM concentration. Screening of aptamer binding to the IgE protein and corresponding signaling effects were performed using crude preparations of aptamers, which might contain trace amounts of uncoupled 2AP, and using sub-stoichiometric amounts of target IgE protein (see FIG. 2). Crude aptamer preparations using DNA synthesis products were sufficient to identify the position that alters the conformation upon binding, even though aptamer samples might have contained contaminating amounts of uncoupled 2-aminopurine molecules. FIG. 2b shows that an aptamer with 2AP at position 18 showed a significant signaling effect upon addition of protein to the solution, as fluorescence of 2-aminopurine increased about 15%. Subsequent assays using PAGE purified aptamer containing 2-aminopurine in this position demonstrated about a 5.6-fold increase in 2AP fluorescence intensity upon binding to IgE at binding saturating concentration (FIG. 2c). This signaling aptamer maintained high specificity to its target protein; its binding affinity was comparable to the affinity of unmodified aptamer.

We subsequently substituted position 18 of the IgE aptamer with other fluorescent nucleotide analogues. An aptamer with 6MAP as a reporter molecule (PAGE purified) showed a fluorescence increase of about 2 times upon addition of binding saturating IgE protein concentration. An aptamer with 3-methylisoxanthopterin (3MI) as a reporter molecule (PAGE purified) showed a fluorescence increase of about 150% upon aptamer binding to IgE protein.

### EXAMPLE 3

### Fluorescent Platelet-Derived Growth Factor B Aptamer

In this example, a signaling aptamer of platelet-derived growth factor B (PDGF-B) [2] was made using secondary DNA structure calculation algorithms to minimize the set of possible modifications for screening.

An aptamer to platelet-derived growth factor B (PDGF B) was previously described in [2]. The sequence of the minimized aptamer is:

(SEQ ID NO: 6)

5′ CAC AGG CTA CGG CAC GTA GAG CAT CAC CAT GAT CCT
GTG 3′.

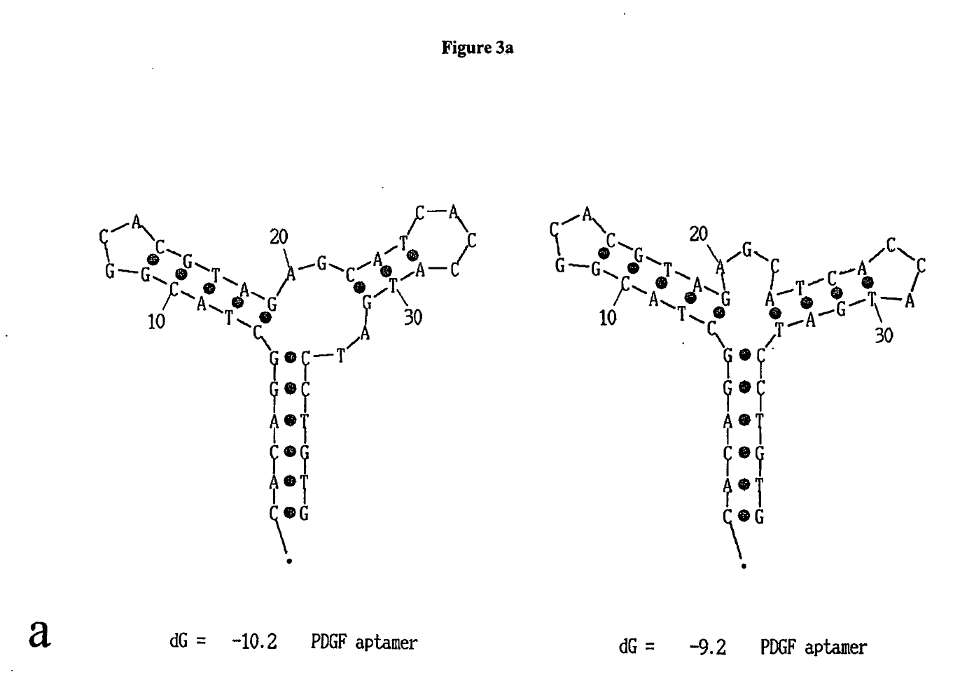

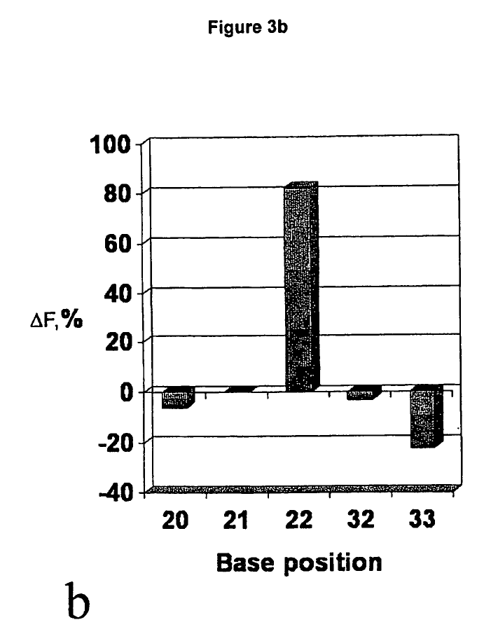

Secondary structure calculations for this aptamer yielded two different possible structures (see FIG. 3a). This suggested that the protein binding site is in the center of the aptamer, at the three helix junction. Therefore, screening of bases which form loops in the center of the aptamer was performed. Five aptamer sequences with 2-aminopurine at positions 20-22 and 32, 33 (i.e., the nucleotide positions at the center of the aptamer) were synthesized and screened for a fluorescence signal change caused by PDGF-B binding to the aptamer. Screening was performed on unpurified aptamers (DNA oligonucleotides were desalted after synthesis) in physiological buffer (20 mM Tris-HCl pH 7.1, 140 mM NaCl, 5 mM KCl, 10 mM CaCl₂, 10 mM MgCl₂) at room temperature (23 C), followed by measuring fluorescence signal changes after addition of 350 nM concentration of PDGF-BB (ie: PDGF-B homo-dimer) protein into unpurified aptamer solution (500 nM) in physiological buffer at room temperature.

The screening assay results (FIG. 3b) showed that position 22 in the aptamer reported binding through an increase in fluorescence. PAGE purified aptamer with 2-aminopurine at position 22 reported binding of PDGF B protein through an approximately 5.8-fold increase in fluorescence of 2-aminopurine. Signaling aptamers were also created using different nucleotide analogs as reporter molecules. For example, PDGF aptamer with 6MAP as a reporter molecule was synthesized and tested; it showed a 6.7-fold increase in fluorescence upon addition of PDGF-B protein to solution. If 3MI is used as a reporter molecule at position 22 of the PDGF-B aptamer, a fluorescence increase of about 2.5 times was observed.

### EXAMPLE 4

Thrombin aptamer with 3MI in the position 7 as described above, was labeled with AlexaFluor™ 430 dye on the 5′ end of polynucleotide using a 21 atom linker:

(SEQ ID NO: 7)

5′ (AlexaFIuor ™ 430)(linker) GGT TGG (3MI)GT GGT
TGG 3′

AlexaFluor™ 430 was covalently attached to the terminal amino group on the 21A Amino linker amidite, which was coupled to the 5′ end of the polynucleotide using standard phosphoramidite DNA synthesis. This particular linker is available from Fidelity Systems, Inc., part number AL21A. (http://www.fidelitysystems.com/AL21A.html)

As described above, the fluorescence signal from 3MI increases upon polynucleotide binding to human α-thrombin protein about 10-fold. When fluorescence of the AlexaFluor™ 430 labeled polynucleotide was measured under the same conditions, 3MI fluorescence signal increased only about 7.5-fold upon addition of human thrombin protein to solution. At the same time, fluorescence signal of AlexaFluor™ 430 increased by about 2.9-fold. AlexaFluor™ 430 fluoresced when no protein is added, due to direct excitation of AlexaFluom™ 430 at 350 nm (excitation wavelength of 3MI). However, the addition of protein in itself did not alter the fluorescence intensity of AlexaFluor™ 430 dye. Control experiments were performed, in which AlexaFluor™ 430 modified polynucleotide fluorescence was excited using 430 nm light (excitation maximum of AlexaFluor™ 430). In this case, no fluorescence from 3MI was observed, as 3MI does not absorb light at 430 nm. Fluorescence spectra of AlexaFluor™ 430 were measured before and after addition of thrombin protein to solution. No signal change was observed after addition of ligand. Therefore, the increase in AlexaFluor™ 430 fluorescence when the sample is excited at 350 nm is due to a FRET effect between 3MI and AlexaFluor 430 molecules.

### EXAMPLE 5

A thrombin aptamer with 3MI in the position 7 was labeled with biotin on the 5′ end of the sequence through a 40 atom linker:

(SEQ ID NO: 8)

5′ (biotin)(linker) GGT TGG (3MI)GT GGT TGG 3′

Biotin, including a 40 atom linker, was attached to the polynucleotide using standard phosphoramidite DNA synthesis to the 5′ end of polynucleotide using DMT-Biotin-Arm34-ACH amidite available from Fidelity Systems, part No. Bt34Ach (http://www.fidelitysystems.com/Bt34ACH.html,).

AlexaFluor™ 430 labeled streptavidin was mixed with 4-times molar excess of biotin labeled aptamers to allow for saturation of steptavidin binding sites. Fluorescence of streptavidin-aptamer complexes was measured before and after addition of human α-thrombin protein to the solution. Fluorescence was measured using 350 nm excitation, which resulted in some direct excitation of AlexaFluor™ 430 as described above. The 3MI fluorescence signal of streptavidin-aptamer complex increases about 7.1-fold when protein is added to solution, while AlexaFluor™ 430 fluorescence increases about 1.9-fold. Control experiments were performed to show that AlexaFluor™ 430 fluorescence signal increase was due to 3MI fluorescence signal increase after protein addition. No AlexaFluor™ 430 fluorescence signal increase was detected in AlexaFluor™ 430-only labeled streptavidin present in solution when thrombin protein is added. No AlexaFluor™ 430 fluorescence signal change was observed upon protein addition if 3MI modified aptamer without the biotin tag was added into the solution. In this case, the complex between streptavidin and aptamer cannot be formed, therefore the 3MI and AlexaFluor™ 430 molecules were not close enough for FRET to be observed.

### REFERENCES CITED

1. Wiegand, T. W., et al., High-affinity oligonucleotide ligands to human IgE inhibit binding to Fc epsilon receptor I. J Immunol, 1996. 157(1): p. 221-30.
2. Green, L. S., et al., Inhibitory DNA ligands to platelet-derived growth factor B-chain. Biochemistry, 1996.35(45): p. 14413-24.
3. Zuker, M., Mfold web server for nucleic acid folding and hybridization prediction. Nucleic Acids Res, 2003.31(13): p. 3406-15.
4. Hawkins, M. E., Fluorescent pteridine nucleoside analogs—A window on DNA interactions. Cell Biochemistry and Biophysics, 2001. 34(2): p. 257-281.

We claim:

1. An improved method for generating fluorescent aptamer polynucleotides, wherein the improvement comprises synthesizing the aptamer sequence with at least one aptamer nucleotide replaced by a fluorescent base analog, wherein the fluorescence intensity of the modified aptamer is detectably altered upon binding to its ligand.

2. The improved method of claim 1 wherein the ligand is a protein.

3. The improved method of claim 1, wherein the fluorescence intensity of the modified aptamer is increased upon binding to its ligand.

4. The improved method of claim 1, wherein placement of the fluorescent base analog is limited by secondary and/or tertiary structural analysis of the aptamer polynucleotide sequence.

5. The improved method of claim 1, further comprising synthesizing the fluorescent aptamer polynucleotide with reference fluorophores.

6. A polynucleotide, comprising a nucleic acid sequence selected from the group consisting of

| | | |
|---|---|---|
| (a) | 5′-GGTTGGXGTGGTTGG-3′; | (SEQ ID NO: 1) |
| (b) | 5′ GGG GCA CGT TTA TCC GTX CCT CCT AGT GGC GTG CCC | (SEQ ID NO: 2) |
| | C 3′; | |
| and | | |
| (c) | 5′ CAC AGG CTA CGG CAC GTA GAG XAT CAC CAT GAT CCT | (SEQ ID NO: 3) |
| | GTG 3′, | |

wherein X is a fluorescent base analog.

7. The polynucleotide of claim 6, wherein X is selected from the group consisting of 2-amino purine (2AP), 3-methyl-isoxanthopterin (3MI), 6-methylisoxanthopterin (6MI), 4-amino-6-methyl-pteridone (6MAP), 4-amino-2,6-dimethyl-pteridone (DMAP), pyrrolo-dC, and 5-methyl-2-pyrimidone.

8. A composition, comprising one or more polynucleotides according claim 5 bound to a solid support.

9. The composition of claim 8, wherein the solid support is selected from the group consisting of microarrays, beads, columns, optical fibers, wipes, nitrocellulose, nylon, glass, quartz, diazotized membranes, silicones, polyformaldehyde, cellulose, cellulose acetate, paper, ceramics, metals, metalloids, semiconductive materials, coated beads, magnetic particles; plastics, gel-forming materials, sol gels, porous polymer hydrogels, nanostructured surfaces, nanotubes, and nanoparticles.

10. The polynucleotide of claim 6, further comprising a one or more of a further fluorescent molecule, an affinity tag, a fluorescent bead, a fluorescent quantum dot, and a chemically reactive linker.

11. A method to detect a ligand of interest, comprising
(a) contacting a sample to be tested with one or more polynucleotides according to claim 5 under conditions to promote binding of the one or more polynucleotides to their relevant ligand;
(b) detecting fluorescence from the one or more polynucleotides; and
(c) correlating an altered fluorescence with the presence of the ligand in the test sample.

12. The method of claim 11, wherein the altered fluorescence comprises an increase in fluorescence.

13. The method of claim 1 wherein the one or more polynucleotides comprises SEQ ID NO: 1, and wherein the ligand is α-thrombin.

14. The method of claim 11, wherein the one or more polynucleotides comprises SEQ ID NO:2, and wherein the ligand is IgE.

15. The method of claim 11, wherein the one or more polynucleotides comprises SEQ ID NO:3, and wherein the ligand is PDGF-B.

16. The method of claim 11, wherein the one or more polynucleotides are bound to a a solid support selected from the group consisting of microarrays, beads, columns, optical fibers, wipes, nitrocellulose, nylon, glass, quartz, diazotized membranes, silicones, polyformaldehyde, cellulose, cellulose acetate, paper, ceramics, metals, metalloids, semiconductive materials, coated beads, magnetic particles; plastics, gel-forming materials, sol gels, porous polymer hydrogels, nanostructured surfaces, nanotubes, and nanoparticles.

17. The method of claim 11, wherein the one or more polynucleotides are in solution during the contacting step.

18. The method of claim 11, wherein the test sample is selected from the group consisting of purified ligand, ligand mixtures, cell lysates, cell culture medium, protein extracts, tissue samples, pathology samples, bodily fluid samples, surface samples, air samples, environmental samples, and animal samples.

19. A method to identify compounds that bind to a ligand of interest, comprising
(a) contacting a polynucleotide according to claim 6 with its ligand under conditions to promote binding of the polynucleotide to the ligand to form a polynucleotide-ligand complex;
(b) detecting fluorescence from the polynucleotide-ligand complex;
(c) contacting the polynucleotide-ligand complex with candidate ligand binding compounds; and
(d) detecting fluorescence of the polynucleotide, wherein a decrease in fluorescence of the polynucleotide following step (c) relative to fluorescence detected from the polynucleotide-ligand complex correlates with the presence of a ligand binding compounds among the candidate ligand binding compounds.


SEQUENCE LISTING (8 sequences)

SEQ ID NO: 1 - DNA, 15 bp, Artificial sequence
  Synthetic oligonucleotide
ggttggngtggttgg

SEQ ID NO: 2 - DNA, 37 bp, Artificial sequence
  Synthetic oligonucleotide
ggggcacgtttatccgtncctcctagtggcgtgcccc

SEQ ID NO: 3 - DNA, 39 bp, Artificial sequence
  Synthetic oligonucleotide
cacaggctacggcacgtagagnatcaccatgatcctgtg

SEQ ID NO: 4 - DNA, 15 bp, Artificial sequence
  Synthetic oligonucleotide
ggttggtgtggttgg

SEQ ID NO: 5 - DNA, 37 bp, Artificial sequence
  Synthetic oligonucleotide
ggggcacgtttatccgtccctcctagtggcgtgcccc

SEQ ID NO: 6 - DNA, 39 bp, Artificial sequence
  Synthetic oligonucleotide
cacaggctacggcacgtagagcatcaccatgatcctgtg

SEQ ID NO: 7 - DNA, 15 bp, Artificial sequence
  Synthetic oligonucleotide
ggttggngtggttgg

SEQ ID NO: 8 - DNA, 15 bp, Artificial sequence
  Synthetic oligonucleotide
ggttggngtggttgg